(19) United States
(12) Patent Application Publication
(10) Pub. No.: US 2024/0148516 A1
(43) Pub. Date: May 9, 2024

(54) METHOD, APPARATUS, AND SYSTEM FOR FACET FUSION

(71) Applicant: UNIVERSITY OF FLORIDA RESEARCH FOUNDATION, INC., Gainesville, FL (US)

(72) Inventor: Sivasupiramaniam SRIHARAN, Gainesville, FL (US)

(21) Appl. No.: 18/576,280

(22) PCT Filed: Jun. 1, 2022
(86) PCT No.: PCT/US2022/031733

§ 371 Date: Jan. 3, 2024

Related U.S. Application Data
(60) Provisional application No. 63/266,656, filed on Jan. 11, 2022.
(60) Provisional application No. 63/236,441, filed on Aug. 24, 2021.
(60) Provisional application No. 63/203,118, filed on Jul. 9, 2021.

(51) Int. Cl. A61F 2/44; A61B 17/70; A61F 2/30; A61F 2/28

(52) U.S. Cl. CPC A61F 2/4405; A61B 17/7059; A61F 2/30749; A61F 2/4455; A61F 2002/2835; A61F 2002/30266; A61F 2002/30579

(57) ABSTRACT

Provided herein is a method, apparatus, and system for a fusion of a facet joint., and more particularly, for a method apparatus and system for facet fusion that provides reliable facet joint stability and requires less invasive surgery. Methods include filling a cavity of a body of a device with a bone grafting material; inserting the body of the device between an inferior facet and a superior facet of a facet joint; and securing the inferior facet to the superior facet using the device.

### CROSS-REFERENCE TO RELATED APPLICATIONS

This application is a national stage entry of PCT Application Serial No. PCT/US2022/031733, filed on Jun. 1, 2022, which claims priority to US Provisional Patent Application Ser. Nos: 63/266,656, filed on Jan. 11, 2022, 63/236,441, filed on Aug. 24, 2021, and 63/203,118, filed on Jul. 9, 2021, the contents of each of which are hereby incorporated by reference in their entirety.

### FIELD

Provided herein is a method, apparatus, and system for facet fusion, and more particularly, for a method apparatus and system for facet fusion that provides reliable facet joint stability and requires less invasive surgery.

### BACKGROUND

Fusion of the facet joint in patients with degenerative disc and facet joint disease is often the ultimate goal of posterior column fusion surgery in the lumbar spine, as opposed to anterior column fixation using various interbody cages and spacers. Lumbar fixation is a type of surgery that uses devices to stabilize vertebrae in the lumbar spine. Posterior lumbar fixation techniques rely on devices such as pedicle screws or interspinous devices for achieving fusion across the facet joint. Pedicle screws are screws that are received into and through vertebrae to provide stabilization and may be attached to rods or other stabilizing devices for lumbar fixation. Because pedicle screws rely on engagement through vertebrae, there is an increased risk of nerve injury and other complications. Interspinous devices do not provide fixation across the facet joint.

With existing pedicle screw fixation, adjacent segment degeneration with stenosis and/or instability is treated by extending the decompression and fusion to the next level with placement of a new pedicle screw at the adjacent segment. This results in greater soft tissue disruption to expose previous hardware and is further complicated by difficulties in trying to connect the new pedicle screw to existing hardware. Current facet fixation devices require relatively invasive surgery and are unreliable, particularly in patients with osteopenic bones.

### BRIEF SUMMARY

Example embodiments of the present invention generally related to fusion of the facet joint, and more particularly, to a method, apparatus, and system to provide facet joint fusion in a manner that is more reliable and less invasive than current techniques. Embodiments provided herein include a method for facet joint fusion including: filling a cavity of a body of a device with a bone grafting material; inserting the body of the device between an inferior facet and a superior facet of a facet joint; and securing the inferior facet to the superior facet using the device. Methods of an example embodiment further include: decorticating a surface of the inferior facet forming a decorticated inferior facet surface; and decorticating a surface of the superior facet forming a decorticated superior facet surface, where the body of the device is inserted between the decorticated inferior facet surface and the decorticated superior facet surface.

According to some embodiments, securing the inferior facet to the superior facet using the device includes: inserting a first fastener through a stabilizing plate of the device into the inferior facet; and inserting a second fastener through the stabilizing plate of the device into the superior facet. Securing the inferior facet to the superior facet using the device includes, in some embodiments, clamping the body of the device between the inferior facet and the superior facet. Methods of an example embodiment further include securing a tapered prism to the stabilizing plate, where the tapered prism is the body of the device.

Embodiments provided herein include a device for facet joint fusion including: a body defining a cavity, where the body is configured to be inserted between an inferior facet and a superior facet of a facet joint; and securing means, where the securing means secures the body to the inferior facet and the superior facet. The securing means of an example embodiment includes two or more teeth rotatably received within the cavity, where the two or more teeth define an insertion position where the two or more teeth do not extend outside of the cavity, and a deployed position, where the two or more teeth are rotated to extend outside of the cavity. In the deployed position of an example embodiment, the two or more teeth engage, on opposite sides of the device, the inferior facet and the superior facet. According to some embodiments, bone graft material is received within the cavity before insertion between the inferior facet and the superior facet.

According to some embodiments, the two or more teeth are attached to a shaft, where the shaft is rotatable within the body, and where the shaft includes a head external to the body of the device for rotating the shaft. The teeth of an example embodiment are rotatable from the insertion position to the deployed position in response to rotation of the head of the shaft. The securing means includes, in some embodiments, a stabilizing plate, a first fastener, and a second fastener, where the first fastener attaches through the stabilizing plate to the superior facet, where the second fastener attaches through the stabilizing plate to the inferior facet.

Embodiments provided herein include a system for facet joint fusion including: a body defining a cavity configured to be inserted between an inferior facet and a superior facet of a facet joint; a first fastener for securing the body, at least indirectly, to the inferior facet; and a second fastener for securing the body, at least indirectly, to the superior facet. The first fastener for securing the body, at least indirectly, to the inferior facet includes at least one first tooth, where the second fastener for securing the body, at least indirectly, to the superior facet includes at least one second tooth. According to some embodiments, the at least one first tooth and the at least one second tooth are attached to a shaft on opposite sides of the shaft.

According to some embodiments, the shaft is rotatably disposed within the cavity of the body, where the at least one first tooth and the at least one second tooth define an insertion position whereby the body is insertable between the inferior fact and the superior facet, and where the at least one first tooth and the at least one second tooth define a deployed position, where the at least one first tooth and the at least one second tooth engage the inferior facet and the superior facet, respectively. The at least one first tooth and the at least one second tooth are rotatable, in some embodiments, from the insertion position to the deployed position in response to rotation of the shaft imparted by rotation of a head of the shaft, where the head is disposed outside of the body.

According to some embodiments, the system further includes a stabilizing plate attached to the body, where the stabilizing plate spans the facet joint, where the first fastener includes a first screw inserted through a first hole of the stabilizing plate and attached to the inferior facet, where the second fastener includes a second screw inserted through a second hole of the stabilizing plate and attached to the superior facet. The stabilizing plate of an example embodiment includes a wedge element, where the wedge element engages the superior facet and secures the superior facet between the body and the stabilizing plate. The stabilizing plate of an example embodiment is adjustably attached to the body, where the body includes a threaded shaft extending from the body, and the stabilizing plate defines a slot through which the threaded shaft is received, where a nut secured on the threaded shaft secures the stabilizing plate to the body.

### BRIEF DESCRIPTION OF THE DRAWINGS

Having thus described the invention in general terms, reference will now be made to the accompanying drawings, which are not necessarily drawn to scale, and wherein:

FIG. 1 illustrates a diagram of a lumbar vertebra according to an example embodiment of the present disclosure;

FIG. 2 illustrates a diagram vertebrae of the lumbar spine according to an example embodiment of the present disclosure;

FIG. 3 illustrates a device for fusion of the facet joint according to an example embodiment of the present disclosure;

FIG. 4 illustrates several views of the device of FIG. 3 for fusion of the facet joint according to an example embodiment of the present disclosure;

FIG. 5 is an image of the device for fusion of the facet joint as installed into a facet joint according to an example embodiment of the present disclosure;

FIG. 6A-B illustrate diagrams of how the device for fusion of the facet joint engages the facet joint according to an example embodiment of the present disclosure;

FIG. 7A-C illustrates components of another device for fusion of the facet joint according to an example embodiment of the present disclosure;

FIG. 8 illustrates the components of FIG. 7A-C assembled as the device for fusion of the facet joint according to an example embodiment of the present disclosure;

FIG. 9 illustrates the device of FIG. 8 inserted into a facet joint according to an example embodiment of the present disclosure;

FIG. 10 illustrates the device of FIGS. 8 and 9 as inserted and installed into a facet joint according to an example embodiment of the present disclosure; and

FIG. 11 is an image of the device for fusion of the facet joint as installed into a facet joint according to an example embodiment of the present disclosure.

### DETAILED DESCRIPTION OF THE DRAWINGS

The present invention now will be described more fully hereinafter with reference to the accompanying drawings, in which some, but not all embodiments of the invention are shown. Indeed, the invention may be embodied in many different forms and should not be construed as limited to the embodiments set forth herein; rather, these embodiments are provided so that this disclosure will satisfy applicable legal requirements. Like numbers refer to like elements throughout.

Embodiments of the present disclosure generally relate to a method, apparatus and system for fusing facet joints to provide stabilization to a lumbar region of a spinal column. Conventional methods of posterior stabilization of the spine uses pedicle screws that rely on achieving fusion across the facet joint. Current techniques of packing the lateral gutters or even interspinous device is less effective in achieving fusion across the facet joint. There are presently no effective devices that allow for adequate stabilization of a facet joint in conjunction with a surface or device structure that allows for good bony fusion of the joint. Embodiments described herein provide a system that fuses facet joints in a manner that is less invasive than current techniques and provides a lower risk of spinal cord injury.

Embodiments of the system for fusing facet joints described herein can be placed via either open surgery, such as after a laminectomy, or percutaneously through a working tube docked on the facet joint. According to an example procedure, the facet joint is exposed and decorticated with a small, round burr down to bleeding bone and to thin the cortical cover. The device of example embodiments is then packed with bone graft and putty and placed in the joint space. The device is then locked in place. Embodiments described herein provide two different devices, detailed further below, that serve the same purpose and can be used for facet joint fusion.

Embodiments described herein provide robust fixation of the facet joint on par with pedicle screw fixation and embodiments can be employed as stand-alone fixation of the spine as detailed further below, or in conjunction with other implants, such as anterior cages, for 360-degree fusions. Embodiments can be employed for stabilization after lumbar laminectomy to prevent post-operative mechanical weakness and decrease the chance of delayed spondylolisthesis. Embodiments can further be used in conjunction with an interspinous devices in patients with spinal stenosis who often have concurrent facet arthritis. This would allow for a complete, minimally invasive decompression and stabilization option with better three-point fixation as well as providing facet fusion. Fusion of the facet joint rather than intertransverse or interspinous fusion allows for a clinically and biomechanically better outcome. Embodiments may be used to back up a posterior lumbar interbody fusion (PLIF) surgery, transforaminal lumbar interbody fusion (TLIF) surgery graft, an extreme lateral interbody fusion (XLIF), or other lateral disc spacer. In such an embodiment, the devices disclosed herein can be placed percutaneously in the lateral or prone position using X-ray imaging alone without requiring any form of intraoperative navigation that is required for the placement of pedicle screws.

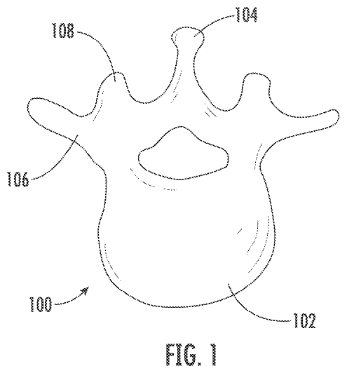

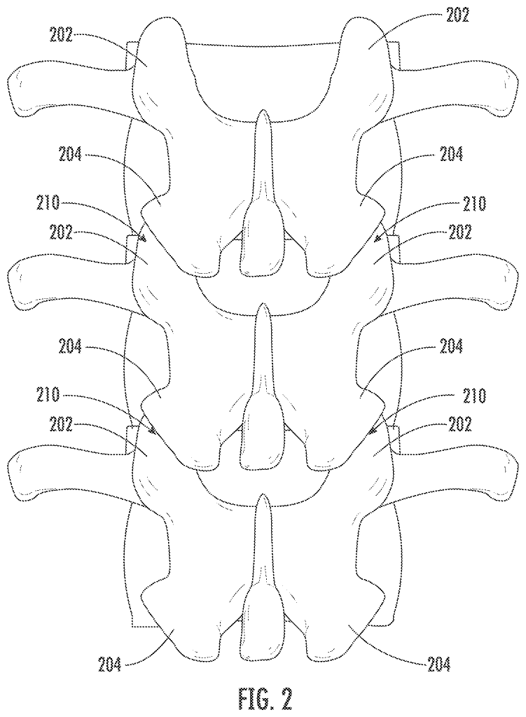

FIG. 1 illustrates elements of a spine including a top-view of a single vertebra 100 including vertebral body 102, laminae 104, pedicle 106, and facet joint 108. FIG. 2 illustrates an anterior view of three vertebra as found attached within the body. Each vertebra includes a pair of superior facets 202 and a pair of inferior facets 204. Between the superior facets 202 and the inferior facets 204 of adjacent vertebra is are facet joints 210. A pair of facet joints 210 join together the facets of adjacent vertebra. While fixation using pedicle screws involves screws passing through the pedicle and into the vertebral body of the spine, embodiments of the facet fusion device described herein is less invasive while providing a structural interface between the inferior facets 204 and superior facets 202 of a facet joint 210.

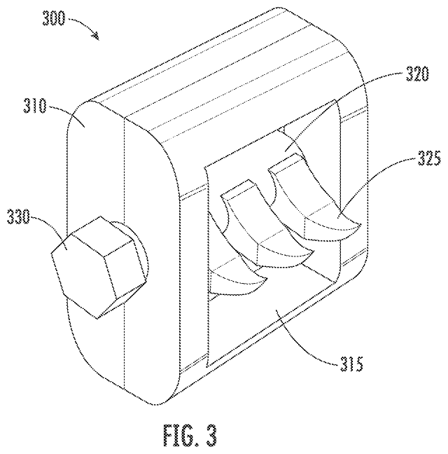

FIG. 3 illustrates an example embodiment of a device for facet fusion as disclosed herein. As shown, the device 300 includes a device body 310 defining a cavity 315. Within the cavity 315 is a shaft 320, rotatable around an axis defined across the cavity. Three teeth 325 extend from the shaft 320 and include an asymmetrical profile with a point of each tooth extending in the same direction as illustrated further below. The shaft 320 with the teeth 325 is rotatable relative to the body 310 through turning of a head 330 of the shaft. The head can include any type of profile, but preferably includes a profile that enables force to be applied to the turning of the shaft for engagement detailed below. The head 330 can be, for example, a hexagonal shape head for engagement with a tool as shown in FIG. 3. Optionally, the head can be flat, knurled, or otherwise textured to enable gripping of the head for turning. Further, the head can have a cavity, such as a keyway for rotation, such as a flat keyway (e.g., for a flat screwdriver), a Philips-head keyway, an Allen-key keyway, a Torx keyway, etc. The body of the device 300 may be, for example, around ten to fifteen millimeters across. However, devices may vary in size based on the specific patient, such that embodiments may be larger or smaller.

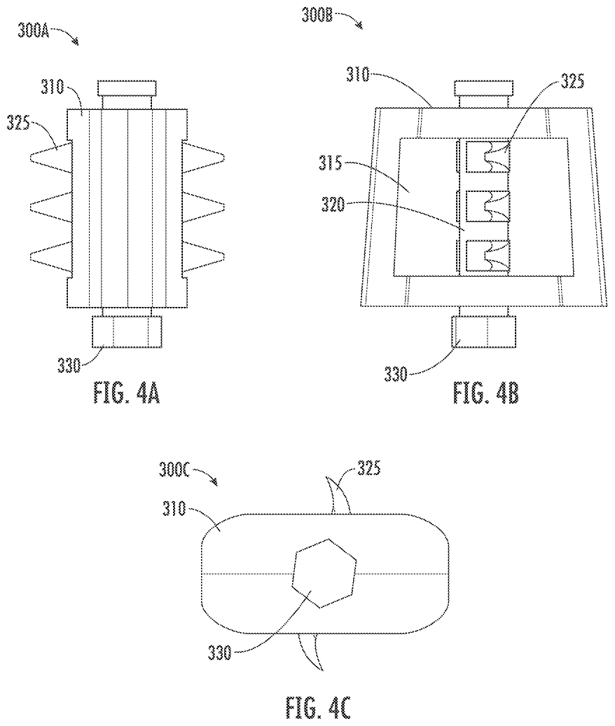

FIG. 4 illustrates additional views of the device 300, including profile view 300A which illustrates the protrusion of the teeth 325 from the body 310. The teeth 325 extend from the body 310 to engage bone as described further below. The head 330 can rotate the teeth 325 from a position where the teeth lay flat within the cavity 315 for insertion, to a position where the teeth extend from the body as shown in the profile view 300A. In the plan view 300B the body 310 is shown with a taper from one end to the other. This taper corresponds to a shape of the facet joint. Embodiments may not include a taper as shown, or may be tapered to a greater degree. The cavity 315 defines a volume into which bone graft is received to facilitate fusion of the facet joint described further below. The cavity 315 is also where the teeth 325 are positioned when the device is inserted into the facet joint. End view 300C illustrates the protrusion of the teeth 325 from the body.

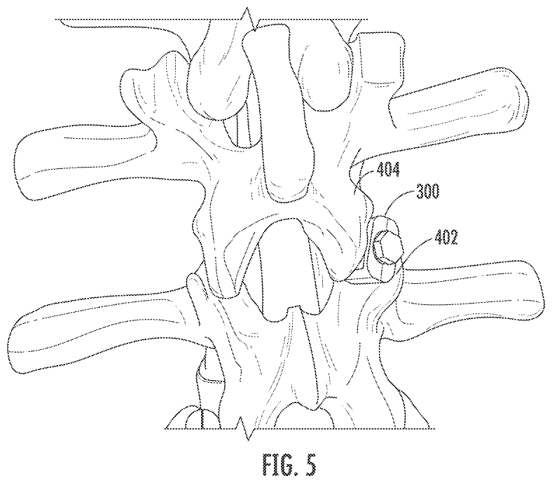

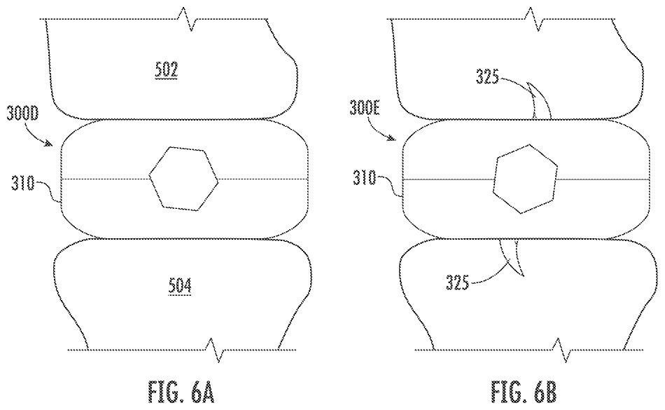

FIG. 5 illustrates an example embodiment of the device 300 inserted into a facet joint. As shown, the device 300 is inserted between the inferior facet 404 and the superior facet 402. The teeth of the device are rotated to an insertion position where the teeth are entirely contained within the cavity of the device in order to insert the device into the facet joint. The cavity is further packed with bone graft, bone putty, or other bone fusion material with the teeth in the insertion position. FIG. 6A illustrates an example embodiment of the facet fusion device 300 in an insertion position 300D with the teeth rotated to within the cavity of the device inserted between an inferior facet 504 and a superior facet 502. FIG. 6B shows the facet fusion device 300 with the teeth 325 in the deployed position 300E, engaging the inferior facet 504 and the superior facet 502. The teeth 325 secure the device 300 within the facet joint and fuses the facet joint with the device. The bone fusion material within the cavity of the device enhances the fusion through stimulation of bone growth through the cavity to achieve permanent fusion of the facet joint.

The facet fusion device described herein can be implanted in a process or method including one or more of the following operations. The device may be implanted in the facet joint at the time of surgery through small incisions over the facet joint. The facet joint is initially exposed and decorticated such that the device is then placed within the joint and the locking mechanism activated. The locking mechanism of the embodiment of FIGS. 3-6B includes the teeth 325 rotated from within the cavity in the insertion position to a deployed position where the teeth engage and lock into the facets of the facet joint. The device of example embodiments can optionally be implanted in a minimally invasive fashion through a working portal that includes a series of progressively larger tissue dilators followed by a cylindrical working tube docked on the facet joint.

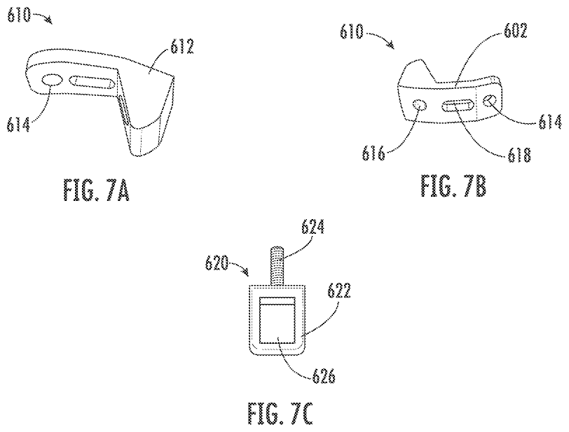

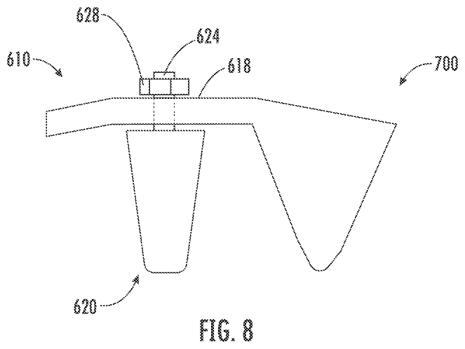

FIGS. 7A-C illustrate components of another facet fusion device including a stabilizing plate 610 having a wedge element 612, the stabilizing plate defining a first fastener hole 614, a second fastener hole 616, and a slot 618. A second component of the facet fusion device is a rectangular, tapered prism 620 with rounded corners 622 and a threaded stem 624. The prism 620 defines a cavity 626. The components of FIGS. 7A-C fit together as illustrated in FIG. 8 with the threaded stem 624 received through slot 618, and a nut 628 to secure the stabilizing plate 610 to the tapered prism 620.

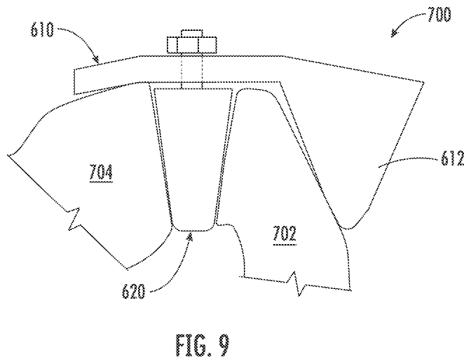

The facet fusion device 700 including the tapered prism 620 and stabilizing plate 610 are used to secure the superior facet to the inferior facet of a facet joint. FIG. 9 illustrates how the facet fusion device 700 engages the facet joint with the tapered prism 620 inserted between the inferior facet 704 and the superior facet 702. The wedge element 612 engages a back side of the superior facet 702, while the tapered prism 620 is movable along the stabilizing plate 610 within the slot 618 of the stabilizing plate to ensure proper fit of the facet fusion device 700 with the specific facet joint to which it is attached.

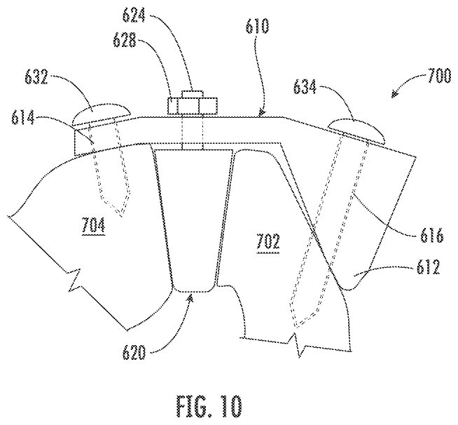

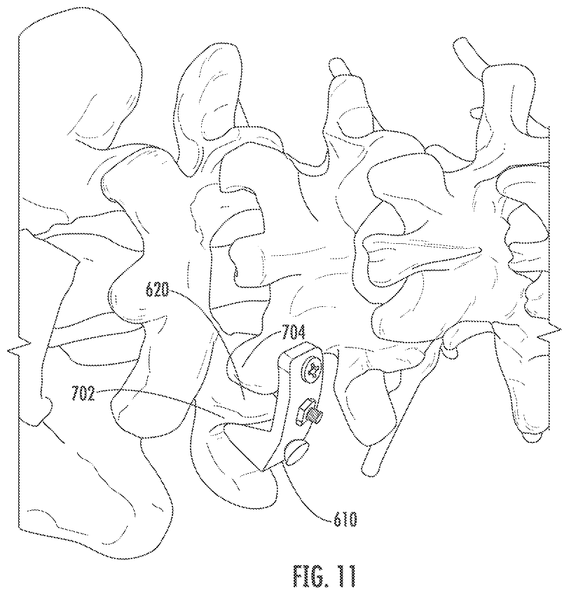

FIG. 10 illustrates the facet fusion device 700 as secured to the facet joint. As shown, a first fastener 632 is inserted through the first fastener hole 614 of the stabilizing plate 610 into the inferior facet 704, while a second fastener 634 is inserted through the second fastener hole 616 into the superior facet 702. The facet fusion device 700, and specifically the wedge element 612 of FIG. 10 can further enable accurate placement of a screw into the pedicle of the lower vertebra through the second fastener hole. Such placement of a screw into the pedicle can provide further purchase of the screw into boney material for stabilization. The tapered prism 620 is secured in place to the stabilizing plate 610 using the threaded stem 624 as the nut 628 is tightened. This securing process effectively clamps the tapered prism 620 in place within the facet joint. The cavity 626 of the tapered prism is filled with a bone fusion material such that bone growth is stimulated to achieve a secure fusion of the facet joint. FIG. 11 illustrates the facet fusion device secured in place including the stabilizing plate 610 spanning the facet joint while the tapered prism 620 is between the inferior facet 704 and the superior facet 702.

The facet fusion device 700 of FIGS. 7A-11 is an example embodiment; however, embodiments can include various shapes of the stabilizing plate 610 and tapered prism 620. For example, the wedge element 612 of the stabilizing plate may take various shapes and forms, and the stabilizing plate may extend over a greater portion of the inferior facet.

The facet fusion device of FIGS. 7A-11 may be implanted via open or minimally invasive surgical techniques using a working portal. To implant the device, the facet bone is exposed and opened. The facet joint is then decorticated with a small bur, such as using an electric or pneumatic drill. The cavity of the body of the prism can be packed with bone graft, bone putty, or a combination thereof before insertion. The prism of the facet fusion device 700 is then tamped into the facet joint and the stabilization plate then attached and fastened to the threaded stem of the prism. A screw is then inserted medially into a superior facet of the spine, while another screw of is inserted into a pedicle of an inferior vertebra of the facet joint. The placement of the screw into the pedicle using the stabilization plate accurately cannulates the pedicle without use of stereotactic navigation, fluoroscopy, or similar methods. The removal of such methods needed to accurately cannulate the pedicle reduces costs and complexity of the surgical procedure.

Embodiments provided herein provide facet stabilization and fusion for effectively fusing the lumbar facet joint, and providing stability and sufficient contact area for the bone fusion mass. Embodiments provide devices capable of implantation to fuse the lumbar facet joint to relieve joint pain. Embodiments are minimally invasive relative to pedicle screws and provide secure fixation with lower risk of complications. Avoiding insertion of pedicle screws reduces the risk of nerve injury, screw pull-out, and failure of instrumentation particularly in osteopenic patients. Further, embodiments do not require placement of a screw at the upper level of the fusion that can potentially compromise the integrity of an adjacent facet joint. This leads to less soft tissue disruption and muscle injury, therefore less blood loss and postoperative pain.

With current pedicle screw fixation, adjacent segment degeneration with stenosis and/or instability is treated by extending the decompression and fusion to the next level with placement of a new pedicle screw at the adjacent segment. This leads to greater tissue disruption to expose previous hardware and is compounded by difficulties in hooking up the new pedicle screw to existing hardware. Using embodiments described herein, the prior hardware does not need to be exposed. Once the laminectomy is performed, the devices of example embodiments described herein can be engaged in the facet joint. Embodiments provide a surface/carrier for bony fusion across the facet joint compared to current lateral gutter/inter transverse fusion or interspinous fusion with interspinous devices. Embodiments disclosed herein provide very secure fixation of the facet joint and vertebra and can replace current alternate pedicle screw fixation.

Embodiments of the facet fusion device of FIGS. 7A-11 provide three points of bone fixation that enables a robust and biomechanically stable fixation of the facet joint and therefore stabilization of vertebrae. Given the multiple points of fixation and the large contact area with the facet joint itself, there is greater load sharing. This load sharing is important in patients with osteopenic bones where there is a greater likelihood of screw pull out and failure of instrumentation in the conventional fusion techniques.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. A method for facet joint fusion comprising:
filling a cavity of a body of a device with a bone grafting material;
inserting the body of the device between an inferior facet and a superior facet of a facet joint; and
securing the inferior facet to the superior facet using the device.

2. The method of claim 1, further comprising:
decorticating a surface of the inferior facet forming a decorticated inferior facet surface; and
decorticating a surface of the superior facet forming a decorticated superior facet surface, wherein the body of the device is inserted between the decorticated inferior facet surface and the decorticated superior facet surface.

3. The method of claim 2, wherein securing the inferior facet to the superior facet using the device comprises:
inserting a first fastener through a stabilizing plate of the device into the inferior facet; and
inserting a second fastener through the stabilizing plate of the device into the superior facet.

4. The method of claim 3, wherein securing the inferior facet to the superior facet using the device comprises clamping the body of the device between the inferior facet and the superior facet.

5. The method of claim 3, further comprising:
securing a tapered prism to the stabilizing plate, wherein the tapered prism is the body of the device.

6. A device for facet joint fusion comprising:
a body defining a cavity, wherein the body is configured to be inserted between an inferior facet and a superior facet of a facet joint; and
securing means, wherein the securing means secures the body to the inferior facet and the superior facet.

7. The device of claim 6, wherein the securing means comprises two or more teeth rotatably received within the cavity, wherein the two or more teeth define an insertion position where the two or more teeth do not extend outside of the cavity, and a deployed position, where the two or more teeth are rotated to extend outside of the cavity.

8. The device of claim 7, wherein in the deployed position, the two or more teeth engage, on opposite sides of the device, the inferior facet and the superior facet.

9. The device of claim 8, wherein bone graft material is received within the cavity before insertion between the inferior facet and the superior facet.

10. The device of claim 7, wherein the two or more teeth are attached to a shaft, wherein the shaft is rotatable within the body, and wherein the shaft comprises a head external to the body of the device for rotating the shaft.

11. The device of claim 10, wherein the teeth are rotatable from the insertion position to the deployed position in response to rotation of the head of the shaft.

12. The device of claim 6, wherein the securing means comprises a stabilizing plate, a first fastener, and a second fastener, wherein the first fastener attaches through the stabilizing plate to the superior facet, wherein the second fastener attaches through the stabilizing plate to the inferior facet.

13. A system for facet joint fusion comprising:
a body defining a cavity configured to be inserted between an inferior facet and a superior facet of a facet joint;
a first fastener for securing the body, at least indirectly, to the inferior facet; and
a second fastener for securing the body, at least indirectly, to the superior facet.

14. The system of claim 13, wherein the first fastener for securing the body, at least indirectly, to the inferior facet comprises at least one first tooth, wherein the second fastener for securing the body, at least indirectly, to the superior facet comprises at least one second tooth.

15. The system of claim 14, wherein the at least one first tooth and the at least one second tooth are attached to a shaft on opposite sides of the shaft.

16. The system of claim 15, wherein the shaft is rotatably disposed within the cavity of the body, wherein the at least one first tooth and the at least one second tooth define an insertion position whereby the body is insertable between the inferior facet and the superior facet, and wherein the at least one first tooth and the at least one second tooth define a deployed position, whereby the at least one first tooth and the at least one second tooth engage the inferior facet and the superior facet, respectively.

17. The system of claim 16, wherein the at least one first tooth and the at least one second tooth are rotatable from the insertion position to the deployed position in response to rotation of the shaft imparted by rotation of a head of the shaft, wherein the head is disposed outside of the body.

18. The system of claim 13, further comprising:
a stabilizing plate attached to the body, wherein the stabilizing plate spans the facet joint, wherein the first fastener comprises a first screw inserted through a first hole of the stabilizing plate and attached to the inferior facet, wherein the second fastener comprises a second screw inserted through a second hole of the stabilizing plate and attached to the superior facet.

19. The system of claim 18, wherein the stabilizing plate comprises a wedge element, wherein the wedge element engages the superior facet and secures the superior facet between the body and the stabilizing plate.

20. The system of claim 18, wherein the stabilizing plate is adjustably attached to the body, wherein the body comprises a threaded shaft extending from the body and the stabilizing plate defines a slot through which the threaded shaft is received, wherein a nut secured on the threaded shaft secures the stabilizing plate to the body.
